(19) United States
(12) Patent Application Publication
(10) Pub. No.: US 2014/0305189 A1
(43) Pub. Date: Oct. 16, 2014

(54) METHOD AND APPARATUS FOR CONTINUOUS MEASUREMENT OF DIFFERENCES IN GAS CONCENTRATIONS

(71) Applicant: Sable Systems International, Inc., Las Vegas, NV (US)

(72) Inventor: John R.B. Lighton, Las Vegas, NV (US)

(21) Appl. No.: 14/313,832

(22) Filed: Jun. 24, 2014

Related U.S. Application Data
(62) Division of application No. 13/765,519, filed on Feb. 12, 2013.
Application No. 13/765,519 is a division of application No. 12/759,197, filed on Apr. 13, 2010, now abandoned.
(60) Provisional application No. 61/253,744, filed on Oct. 21, 2009.

(51) Int. Cl. G01N 33/497; G01N 33/00

(52) U.S. Cl. CPC G01N 33/497; G01N 33/0009
USPC 73/23.3; 73/31.05

(57) ABSTRACT

A method for continuous measurement of differences in gas concentrations, comprises providing at least first and second gas analyzers, connecting a stream of incurrent fluid to a chamber containing an animal, withdrawing air from the chamber to form a stream of excurrent fluid, taking first subsamples of the excurrent fluid in a first subsampler, taking a subsample of the incurrent fluid in a second subsampler, alternately providing excurrent fluid from the first subsampler to the first gas analyzer and to the second gas analyzer to measure the gas concentrations in the excurrent fluid, and alternately providing incurrent air from the second subsampler to the first gas analyzer and to the second gas analyzer to measure the gas concentrations in the incurrent fluid.

### CROSS-REFERENCE TO RELATED APPLICATIONS

The present application is a divisional and claims the priority benefit of U.S. patent application Ser. No. 12/759,197 filed Apr. 13, 2010, which claims the priority benefit of U.S. provisional application No. 61/253,744 filed Oct. 21, 2009 and U.S. provisional application No. 61/168,846 filed Apr. 13, 2009, the disclosure of which is incorporated herein by reference.

### BACKGROUND OF THE INVENTION

1. Field of the Invention

The present invention relates to measurement of gas concentrations. More particularly, the present invention relates to continuous measurement of differences in gas concentrations.

2. Description of the Related Art

Turning for purposes of illustration to a typical embodiment of the invention in the field of respirometry, it is noted that respirometry is defined, in practice, as the measurement of gas exchange rates of organisms or substances via changes in gas concentrations in the liquid or gaseous medium surrounding the organisms or substances. Various modalities of this art exist, of which flow-through respirometry, sometimes referred to as indirect calorimetry, is the most common in general practice. Flow-through respirometry depends upon the accurate measurement of at least two parameters; one or more gas concentration changes caused by the organisms or substance, and the flow rate of the liquid or gaseous medium. The gas exchange rate of an organism or substance is calculated from the difference that an organism or substance causes in the gas concentration within the liquid or gaseous medium flowing past the organism or substance.

Where the organism or substance consumes a given species of gas, such as oxygen by way of example, this difference in gas concentration is commonly expressed as FiO.sub.2—FeO.sub.2, where FiO.sub.2 is the fractional concentration of O.sub.2 within the incurrent gas stream prior to contact with the organism or substance, and FeO.sub.2 is the fractional concentration of O.sub.2 within the excurrent gas stream after such contact. Where the organism or substance produces a given species of gas, such as carbon dioxide by way of example, this difference in gas concentration is commonly expressed as FeCO.sub.2—FiCO.sub.2, where FeCO.sub.2 is the fractional concentration of CO.sub.2 within the excurrent gas stream after contact with the organism or substance, and FiCO.sub.2 is the fractional concentration of CO.sub.2 within the incurrent gas stream prior to such contact. In combination with knowledge of the flow rate of the liquid or gaseous medium, measured prior to or after contact with the organism or substance, it is possible to calculate the rate of gas exchange of the organism or substance using well-established equations, which are summarized in the textbook “Measuring Metabolic Rates: A Manual for Scientists”, by John Lighton (Oxford University Press, 2008).

Assuming that incurrent gas concentrations are constant, and that flow rates are accurately measured, the primary source of error in respirometry arises from the gas analyzers, which drift to a greater or lesser extent. In order to mitigate the effects of analyzer drift, it is essential that regular and preferably frequent measurements of incurrent gas concentrations be made. Making measurements of incurrent gas concentrations is usually referred to as “baselining.” When a “baseline” measurement is made, for example of the fractional O.sub.2 concentration of dry incurrent air, the gas analyzer can be adjusted so that its reading equals the known fractional concentration of O.sub.2 in the incurrent air; in this example, typically 0.2094 in a well-ventilated room. This is often referred to as “spanning” the analyzer; in most cases the terms “spanning” and “baselining” are equivalent and are so used here. Other gases of interest in respirometry, notably CO.sub.2 and H.sub.2O, are generally more variable in concentration, and in their cases, “baselining” more frequently refers to the simple measurement of incurrent concentrations with less concern for compensating for analyzer drift.

Switching of the analyzed stream between incurrent and excurrent gas flows is usually accomplished by a manual bypass, or by a manually or automatically operated solenoid valve. Shortly after measurement of incurrent gas concentrations, and after returning to the measurement of excurrent gas concentrations, the highest accuracy of respirometric gas analysis is attained because both incurrent and excurrent gas concentrations were both recently measured. However, as time passes, errors accumulate because analyzer drift causes the measured excurrent gas concentrations to deviate from their true value. To a large extent this “error creep” can be mitigated by re-measuring baselines periodically, and by applying a linear or non-linear correction between successive baseline measurements as described in “Measuring Metabolic Rates: A Manual for Scientists”, by John Lighton (Oxford University Press, 2008). However, periodic baselines disrupt the measurement each time they are taken. The result is a conflicting tradeoff between accuracy of measurement, and disruption of measurement; the more accurate a measurement is required to be, the more it must be disrupted by baselining. This is a universal problem throughout the field of respirometry and other applications requiring the accurate analysis of differential gas concentrations.

Another technique for mitigating analyzer drift errors is to use a differential or dual-absolute gas analyzer. In this case, two equivalent analyzers measure the same gas species, or alternately measure gas concentrations in incurrent and excurrent flows. In the simplest form, this method allows analyzer drift to be reduced but not eliminated. Periodic baselines do not need to be measured as often, but they are still required and still disrupt the respirometric data. In a more complex form, described in Stephens et al. (B B Stephens, P S Bakwin, P P Tans, R M Teclaw, D D Baumann “Application of a Differential Fuel-Cell Analyzer for Measuring Atmospheric Oxygen Variations,” Journal of Atmospheric and Oceanic Technology, 2007), each channel of a dual-absolute gas analyzer is switched alternately between incurrent and excurrent air streams, such that a “square wave” of gas concentrations is produced, the magnitude of which is double that of the absolute (incurrent minus excurrent) gas concentrations. While this method offers excellent sensitivity and is intrinsically drift-free if switching is performed at a high enough frequency, it still disrupts the gas readings at a relatively high rate (minutes to tens of minutes) and so is not suitable for respirometric recordings that require high temporal resolution.

In particular, all of the abovementioned methods are especially problematic if mathematical response compensation for wash-out artifacts must be performed upon the data. Such “instantaneous” compensations are required to compensate for the interactions between the volumes of respirometric chambers, and the rate at which gas or liquid flows through them. These interactions cause rapid changes in gas exchange rates to be “blunted” by the respirometry system. This is a particular problem, for example, in “room calorimeters”, which are large chambers in which the metabolic rates of human volunteers are studied. To compensate for such response distortions, usually the first derivative of the gas concentration signal is multiplied by a constant that is empirically derived for a given system, and that product is added back to the gas concentration signal. Because this technique employs first derivatives, it is sensitive to any abrupt changes in the gas signal, especially such as those caused by baselining Baselining therefore causes massive artifacts in response-corrected respirometry data. Such artifacts can be mitigated by interpolation across baselines, but data are still lost in the process.

### SUMMARY OF THE CLAIMED INVENTION

The present invention relates to novel methods for determining differences in gas concentrations. The method of the present invention yield recordings of gas concentration differences that are not interrupted by the periodic “baselining” and calibrations that are required to measure gas concentration differences with high accuracy. In a typical embodiment, this novel method may be used in respirometry, whether aerial or aquatic, but its utility is by no means limited to that field. It could also, for example, be used for determining the mix ratios of combustible gases to atmospheric oxygen.

According to the present invention, a plurality of gas analyzers (of any kind) are used in a synchronized fashion, such that baselining is interleaved, and the (incurrent versus excurrent gas concentrations) data are combined in real time or during later analysis such that a continuous, uninterrupted, and maximally accurate data vector is created. The data vector thus created contains the difference in gas concentrations between the incurrent and excurrent fluid streams, with minimization of “error creep”, and without intermediate changes of magnitude caused by baselining. The result is a highly accurate and continuous record of gas concentration differences between incurrent and excurrent fluid streams, caused for example by the metabolic uptake or production of gases by an organism or substance, or by the addition of a gas species to the fluid stream, without disruptions. At the same time as allowing the real-time or delayed generation of the combined difference data vector, this method allows for the storage of all raw data, from which the continuous record can be reconstructed, for validation or auditing purposes.

### BRIEF DESCRIPTION OF THE DRAWINGS

FIG. 1 is a diagram illustrating a method according to the present invention in the environment of a single respirometry chamber.

FIG. 2 is a diagram illustrating a method according to the present invention in the environment of another single respirometry chamber.

FIG. 3 is a diagram illustrating a non-limiting example of the operation of the system, when used to analyze a single subject.

FIG. 4 is a diagram illustrating a typical procedure for combining gas concentration recordings by way of non-limiting example.

FIG. 5 is a diagram illustrating recording the signals from the analyzers in “raw data” form and post-processing the analyzer signals to obtain a better combined channel with lower errors.

### DETAILED DESCRIPTION

Persons of ordinary skill in the art will realize that the following description of the present invention is illustrative only and not in any way limiting. Other embodiments of the invention will readily suggest themselves to such skilled persons.

In broad terms, the method of the present invention may employ at least two gas analyzers for each gas species being measured. A typical measurement cycle for any given gas species proceeds as follows. For clarity of description, no elimination of water vapor dilution via chemical water vapor scrubbing or other dilution compensation processes (in the case of gaseous fluid measurements) is included in this description; however, such methods are in practice required in all gaseous gas analysis systems.

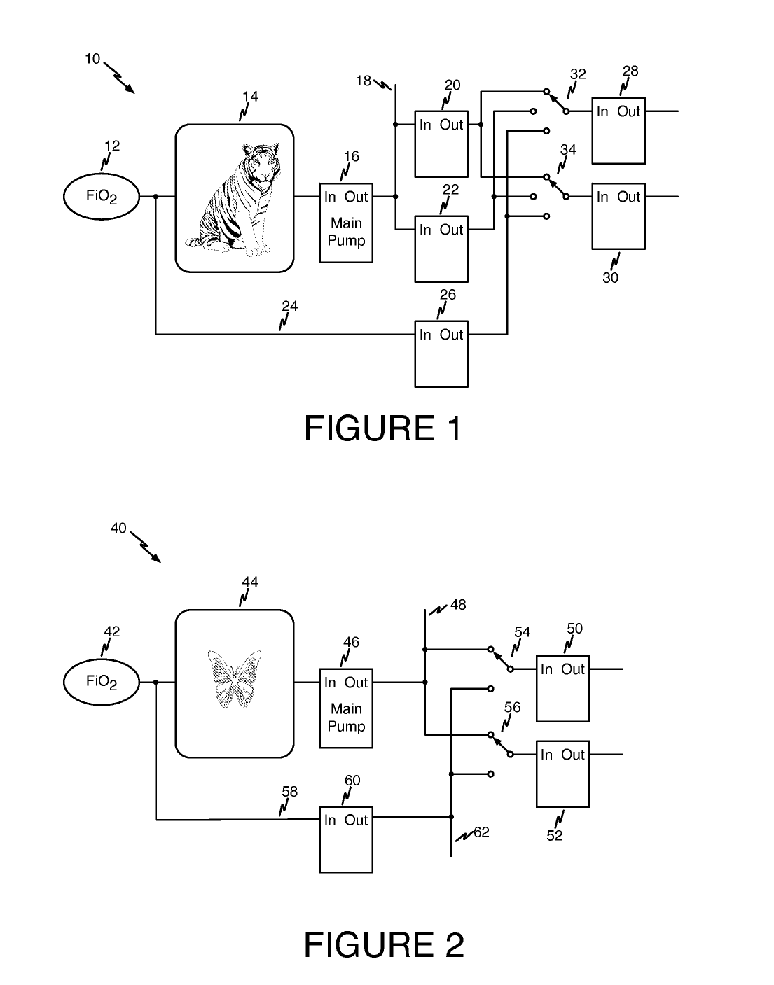

Referring now to FIG. 1, a first exemplary embodiment of a method according to the present invention is shown using a system 10 including a single respirometry chamber or mask. Incurrent air, indicated at reference numeral 12, having fractional O.sub.2 concentration FiO2 is connected to a chamber 14 containing an animal. Air is withdrawn from the chamber 14 by a pump 16. As persons of ordinary skill in the art will appreciate, in alternative embodiments, air may be pushed through the chamber by a pump placed before the chamber in the flow sequence. Most of the excurrent air may be vented as shown at reference numeral 18 but subsamples of the excurrent airstream are taken by two independent subsampling pumps 20 and 22. Persons of ordinary skill in the art will readily observe that alternatively, pressure-based subsampling could be implemented, eliminating the need for subsampling pumps.

Meanwhile a subsample of the incurrent air 12 is taken through tubing 24 into subsampling pump 26. The two gas analysis systems 28 and 30, which in a typical embodiment could be analyzer chains consisting of water vapor, oxygen and carbon dioxide analyzers, can each select the gas flow from any of the subsampling pumps 20, 22, and 26, using multi-way valves or switches 32 and 34, which may by way of example be manual, electronic or pneumatic in operation. In one embodiment valves or switches 32 and 34 may be controlled via a computer or other device such as a state machine that orchestrates their switching, together with recording and processing of flow rate and gas concentration data. The flow rates from the subsampling pumps 20, 22, and 26, are kept identical as far as possible to minimize measurement errors between the three different subsampled air streams. In a typical embodiment, the air streams would be vented from the analyzers after measurement as is known in the art.

Referring now to FIG. 2, another and somewhat simpler exemplary embodiment of the method is shown using a system 40 having a single respirometry chamber. This example assumes that gas analyzers or analyzer chains used each incorporate a flow generation system that can pull air through them to be analyzed. Incurrent air 42 with fractional O.sub.2 concentration FiO2 is connected to a chamber 44 containing an animal. Air is withdrawn from the chamber by a pump 46. In an alternative instantiation, incurrent air may be pushed through the chamber by a pump placed before the chamber in the flow sequence. Most of the excurrent air may be vented as shown at reference numeral 48 but subsamples of the excurrent airstream may sampled by either or both gas analysis chains 50 and 52, by suitable selection of switches 54 and 56, which may by way of example be manual, electronic or pneumatic in operation. Meanwhile a subsample of the incurrent air 12 is taken through tubing 58, optionally into subsampler 60, and is selectable via switches or valves 54 and 56 as an input to either or both gas analysis chains 50 and 52. Excess subsampled incurrent air may optionally be vented as shown at reference numeral 62. In one embodiment, switches 54 and 56 may be controlled via a computer or other device such as a state machine that orchestrates their switching, together with recording and processing of flow rate and gas concentration data. The flow rates from the subsamplers, whether external or intrinsic to the analyzers, are kept identical as far as possible to minimize measurement errors between the different subsampled air streams. In a typical embodiment, the air streams would be vented from the analyzers after measurement.

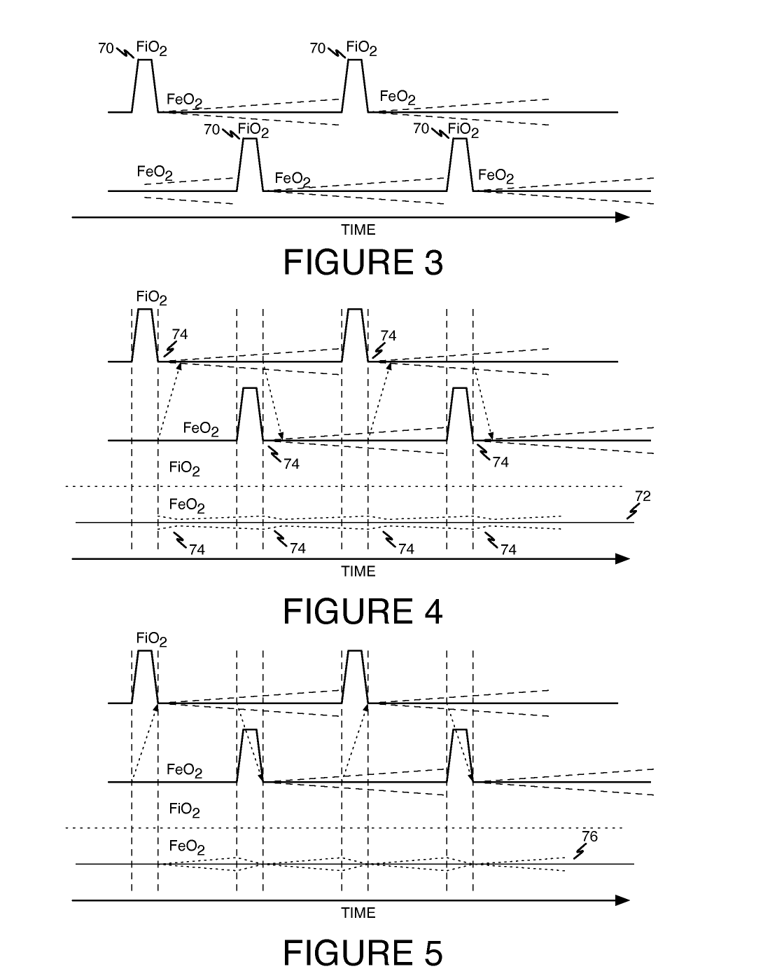

Referring now to FIG. 3, a diagram illustrates a non-limiting example of the operation of the system, when used to analyze the difference between a single incurrent fluid stream and a single excurrent fluid stream. The two horizontal lines are gas concentrations, in this example oxygen, over time, as measured by gas analyzers or gas analyzer chains shown in either FIG. 1 or 2. As can be seen, the two analyzers measure baselines (shown at reference numeral 70) alternately, using the opportunity to calibrate themselves to FiO2 by way of example. Their accuracy is optimal shortly after measuring baselines, as shown by the dotted error curves. The air streams from the subsamplers 28 and 30 of FIG. 1 or from subsampled excurrent air pulled by flow generators intrinsic to the gas analyzer chains of FIG. 2 are likewise alternately measured. It is now possible to combine these two analyzer signals to create a single, continuous recording of, for example, (FiO2—FeO2) by way of non-limiting illustration. Similarly, continuous recordings of, for example, carbon dioxide or water vapor enrichment may be generated in a like fashion.

FIG. 4 illustrates a typical procedure for combining the gas concentration recordings. The combined record 72 is shown at the bottom of the Figure. The periods of maximal accuracy for the gas signals from analyzers 28 and 30 or 50 and 52 of FIGS. 1 and 2, and in the combined trace are shown at reference numeral 74. In this instance the combined trace may be created in real time. The dotted arrows denote the transfer from the concentration output of analyzer 28 (or 50) to the concentration output of analyzer 30 (or 52) and vice versa, at the time of maximum accuracy of the signal to which transfer is made, soon after baselining, in order to create combined channel 72, which consists of the coordinated transfer of the concentration outputs of the two analyzers to create a continuous record of the difference between incurrent and excurrent gas concentrations. Such transfers could be made by simple switching, or preferably, by a graded transfer in which the two analyzer signals are mixed, effecting a gradual transfer that does not produce a significant disruption in combined signal 72. Normalization of the endpoints could optionally take place to ensure minimal disruption.

Because the signals from the analyzers could and should be recorded in “raw data” form, it is also possible to obtain a more accurate combined channel with lower errors by post-processing the analyzer signals. This procedure is shown in FIG. 5. The combined record 70, created by post-processing, is shown at the bottom of the Figure. It can be seen that the estimated error envelope (dotted lines) in combined channel 74, which denotes the periods of maximal accuracy for the gas signals from the analyzer chains and in the combined trace, is smaller than in FIG. 4. The dotted arrows denote the coordinated transfer, effected during post-acquisition processing, of the concentration output data from analyzer 28 (or 50) to 30 (or 52) and vice versa in order to create combined channel 76, which is an optimally accurate and continuous record of the difference between incurrent and excurrent gas concentrations.

The transfer from one analyzer's signal to the other analyzer's signal takes place, immediately after baselining, at the time of maximum accuracy of the signal to which transfer is made. In this case it will be noted that the transfer may begin to take place during the baselining event itself, because the endpoint of the signal following baselining is already known (this is not the case in a real-time generation of the combined channel 72 as in FIG. 4). This facilitates normalization of the endpoints at the point of switch-over. Such normalization could optionally take place to ensure minimal disruption of combined trace 76, especially where significant wash-out compensation is anticipated, as by way of non-limiting example, in room calorimetry.

Scaling the present invention to allow for continuous measurement of multiple animals is simple, and allows the generation of uninterrupted metabolic data in applications such as the metabolic screening of multiple animals such as mice. For optimal results, with frequent baselining, two or more gas analysis chains are required for each experimental subject or excurrent gas stream; A>=N*2, where A is the number of analyzers and N is the number of experimental subjects or excurrent gas streams to be analyzed.

Two analyzer chains allow a roughly equal allocation between measurement and baselining, which yields excellent accuracy. If required, however, a smaller number of analyzer chains can be used if it is acceptable to baseline less frequently. In such cases, typically and by way of non-limiting example, one analyzer chain might be assigned primarily to each experimental subject, and when it was necessary to baseline that analyzer chain (i.e. measure incurrent concentrations), an additional analyzer chain would be allocated to allow measurement of excurrent concentrations during the period when the primary analyzer was baselining When baselining of the primary analyzer is complete, the primary analyzer returns to measuring excurrent concentrations, after which the additional analysis chain can be baselined again and then reassigned to measure the excurrent air from a different chamber, the primary analysis chain of which is about to be baselined. Thus, the number of analyzer chains can be reduced by diminishing the proportional duration for which another analyzer measures excurrent gas concentrations. The absolute minimum number of analyzer chains, A, required to yield continuous recordings of metabolic rate is A=(N+1). In multiple-animal systems (N>1), a cost-benefit analysis will be required to determine A, where generally, (N+1).ltoreq.A.ltoreq.(N*2).

While embodiments and applications of this invention have been shown and described, it would be apparent to those of ordinary skill in the art that many more modifications than mentioned above are possible without departing from the inventive concepts herein. The present invention, therefore, is not to be restricted except in the spirit of the appended claims.

1-26. (canceled)

27. A method and for continuous measurement of differences in gas concentrations, comprising:
providing a plurality of gas analyzers numbering from about (N+1) to about 2N;
providing one or more streams of incurrent gas to multiple chambers;
withdrawing gas from the multiple chambers to form multiple excurrent gas streams of the number N to be analyzed;
taking first subsamples of the excurrent gas from one or more of the N excurrent gas streams in a first subsampler;
taking a subsample of the incurrent gas from the one or more streams of incurrent gas in a second subsampler;
alternately providing excurrent gas from the first subsampler to a first gas analyzer of the plurality of gas analyzers provided and to a second gas analyzer of the plurality of gas analyzers provided to measure the gas concentrations in the excurrent gas; and
alternately providing incurrent gas from the second subsampler to the first gas analyzer of the plurality of gas analyzers provided and to the second gas analyzer of the plurality of gas analyzers provided to measure the gas concentrations in the incurrent gas.

28. The method of claim 27 wherein flow rates from the first and second subsamplers are maintained substantially identical.

29. The method of claim 27 wherein the step of providing a plurality of gas analyzers numbering from about (N+1) to about 2N further comprises providing at least first and second gas analyzer chains.

30. The method of claim 27 wherein the step of providing a stream of incurrent gas to a chamber comprises providing a stream of incurrent gas to a chamber containing an object that consumes or produces gases.

31. The method of claim 30, wherein the step of providing a stream of incurrent gas to a chamber containing an object that consumes or produces gases comprises providing a stream of incurrent gas to a chamber containing an inanimate object that consumes or produces gases.

32. The method of claim 30, wherein the step of providing a stream of incurrent gas to a chamber containing an object that consumes or produces gases comprises providing a stream of incurrent gas to a chamber containing living organism.

33. The method of claim 27 wherein the step of providing one or more streams of incurrent gas to multiple chambers comprises providing one or more streams of incurrent gas to a respiration mask worn by a subject; and withdrawing gas from the multiple chambers to form multiple excurrent gas streams of the number N to be analyzed comprises withdrawing gas from the respiration mask.

34. The method of claim 27 further comprising the step of recording of flow rate and gas concentration data.

35. The method of claim 34, wherein the step of recording of flow rate and gas concentration data comprises continuously recording flow rate and gas concentration data from at least one of all of the first and second gas analyzers and from the one or more streams of incurrent gas.

36. The method of claim 35, wherein the step of recording of flow rate data comprises continuously recording flow rate data from the one or more streams of excurrent or incurrent gas.

37. The method of claim 35, further comprising the step of alternately processing flow rate and gas concentration data from the first and second gas analyzers in real time in order to create a continuous record in real time of the difference in gas concentrations between incurrent and excurrent gas streams.

38. The method of claim 35, further comprising the step of alternately processing pre-recorded flow rate and gas concentration data from the first and second gas analyzers in order to create a continuous record of the difference in gas concentrations between incurrent and excurrent gas streams.

39. The method of claim 27 further comprising withdrawing gas from at least one gas stream source other than a chamber to form multiple excurrent gas streams of number N to be analyzed.

40. The method of claim 39, wherein providing at least first and second gas analyzers comprises providing at least (N+1) gas analyzers comprises providing between about at least (N+1) and about 2N gas analyzer chains.

41. A method and for continuous measurement of differences in gas concentrations, comprising:
providing a plurality of gas analyzers numbering from about (N+1) to about 2N;
providing one or more streams of incurrent gas to one or more chambers;
withdrawing gas from the one or more chambers and at least one gas source other than one of the one or more chambers to form multiple excurrent gas streams of the number N to be analyzed;
taking first subsamples of the excurrent gas from one or more of the N excurrent gas streams in a first subsampler;
taking a subsample of the incurrent gas from the one or more streams of incurrent gas in a second subsampler;
alternately providing excurrent gas from the first subsampler to a first gas analyzer of the plurality of gas analyzers provided and to a second gas analyzer of the plurality of gas analyzers provided to measure the gas concentrations in the excurrent gas; and
alternately providing incurrent gas from the second subsampler to the first gas analyzer of the plurality of gas analyzers provided and to the second gas analyzer of the plurality of gas analyzers provided to measure the gas concentrations in the incurrent gas.

42. The method of claim 41 wherein flow rates from the first and second subsamplers are maintained substantially identical.

43. The method of claim 41 wherein the step of providing a plurality of gas analyzers numbering from about (N+1) to about 2N further comprises providing at least first and second gas analyzer chains.

44. The method of claim 41 further comprising the step of recording of flow rate and gas concentration data.

45. The method of claim 44, wherein the step of recording of flow rate and gas concentration data comprises continuously recording flow rate and gas concentration data from at least one of all of the first and second gas analyzers and from the one or more streams of incurrent gas.

46. The method of claim 45, wherein the step of recording of flow rate data comprises continuously recording flow rate data from the one or more streams of excurrent or incurrent gas.
